(19) United States
(12) Patent Application Publication
(10) Pub. No.: US 2026/0045332 A1
(43) Pub. Date: Feb. 12, 2026

(54) BAYESIAN NETWORK-BASED RISK PROJECTION FOR IMPLANTABLE MEDICAL DEVICES

(71) Applicant: Medtronic, Inc., Minneapolis, MN (US)

(72) Inventors: Yan Liu, Maple Grove, MN (US); Kiran Kuppuswamy, Mounds View, MN (US)

(21) Appl. No.: 19/296,273

(22) Filed: Aug. 11, 2025

Related U.S. Application Data
(60) Provisional application No. 63/682,185, filed on Aug. 12, 2024.

(51) Int. Cl. G16H 10/60; G16H 20/40; G16H 40/67; G16H 50/30

(52) U.S. Cl. CPC G16H 10/60; G16H 20/40; G16H 40/67; G16H 50/30

(57) ABSTRACT

An example system includes a memory configured to store cardiac data associated with a patient population within a Bayesian network structure describing cardiac health events for the patient population. The system may apply new patient data to an Artificial Intelligence model (AI model) trained using the Bayesian network structure to output a risk-benefit assessment specific to a particular patient implantable medical device (IMD). The output may include selectable configuration recommendations and a corresponding risk probability for each configuration. Subsequent to the output, the system may receive clinician input selecting one of the configuration recommendations for the patient IMD. Responsive to receipt of the clinician input, the system may configure delivery of a therapy via the patient IMD using the one of the multiple configuration recommendations selected according to the clinician input.

### RELATED APPLICATIONS

This application claims the benefit of U.S. Provisional Application Ser. No. 63/682,185, filed Aug. 12, 2024, the entire contents of each of which are incorporated herein by reference.

### TECHNICAL FIELD

The disclosure relates generally to medical device systems and, more particularly, medical device systems configured to evaluate patient risk associated with implantable medical devices (IMDs).

### BACKGROUND

Medical devices may be used to monitor physiological signals of a patient and sense certain health events and deliver treatment in response to those health events. For instance, some medical devices are configured to sense and treat abnormal heart rhythms (ventricular arrhythmias) such as ventricular tachycardia and ventricular fibrillation. These life-threatening rhythms can cause sudden cardiac arrest (SCA), which is lethal if not treated. Treatments for such rhythms may include antitachycardia pacing (ATP) and/or antitachyarrhythmia shock, e.g., cardioversion or defibrillation. Treatments for such rhythms may be organized as sequences, where a next therapy in a sequence may be delivered when a previous therapy is determined to be unsuccessful. Some medical devices may be configured to deliver cardiac resynchronization therapy (CRT) pacing to synchronize contraction and improve function of the heart.

### SUMMARY

In general, aspects of this disclosure are directed to techniques for generating a risk assessment for output to a clinician utilizing Bayesian Network models trained for quantifying and predicting risk of therapy delivery for various types of Implantable Medical Devices (IMDs) including Implantable Cardioverter Defibrillator (ICD) and Cardiac Resynchronization Therapy Defibrillator (CRT-D) type medical devices. The risk assessment may be generated specifically for the risk to a particular patient, for a particular patient's IMD, for a particular IMD configuration to be applied to the patient's IMD, for a specific type of IMD if implanted into the particular patient, for a specific IMD product if implanted into the particular patient, or some combination thereof. The risk assessment generated may additionally provide as output to a clinician, multiple recommended configurations for a patient IMD with a corresponding risk for each of the recommended configurations. Additional information may be provided as output to the clinician to aid the clinician in selecting a configuration to apply to a patient IMD including, for example, a risk comparison of a given configuration as applied to the particular patient with a patient population having similar or overlapping health characteristics. Responsive to the clinician selecting one of the recommended configurations, the system may configure the patient's IMD with the clinician selected configuration.

Unlike previous techniques which quantify risk for a patient population or some curated patient subpopulation, the system is configured to not only quantify risk for the patient population and/or patient subpopulation, but additionally determine a customized risk calculation for each individual patient and/or each individual device, given various device/patient inputs and/or unique use conditions and observed events for the patient or device evaluated. Modeling results such as patient/device risk assessments and patient/device risk comparisons with a larger population support clinician decision-making with highly-customized patient/device management recommendations unique to the patient in the context of existing clinical trial data and existing field data. In related examples, modeling results provided by the trained Bayesian Network models may be utilized for optimizing IMDs before product launch.

In one example, this disclosure describes a system comprising a memory configured to store cardiac data associated with a patient population within a Bayesian network structure. In such an example, the cardiac data describes cardiac health events for the patient population. The system also includes processing circuitry in communication with the memory, wherein the processing circuitry is configured to receive new patient data describing at least one of a new cardiac health event determined by a patient implantable medical device (IMD), a record of an arrhythmia treatment applied by the patient IMD, or a programming configuration for the patient IMD. According to such an example, the processing circuitry is further configured to output a risk-benefit assessment specific to the patient IMD, wherein the risk-benefit assessment indicates multiple configuration recommendations for the patient IMD and a corresponding risk probability for each of the multiple configuration recommendations based at least in part on application of the new patient data to an Artificial Intelligence model (AI model) trained using the Bayesian network structure to describe cardiac health profiles for a patient population. Subsequent to output of the risk-benefit assessment specific to the patient IMD, the processing circuitry may receive clinician input with a selection of one of the multiple configuration recommendations for the patient IMD. Responsive to receipt of the clinician input with the selection of the one of the multiple configuration recommendations for the patient IMD, the processing circuitry may configure delivery of a therapy via the patient IMD using the one of the multiple configuration recommendations selected according to the clinician input.

In another example, this disclosure describes a method comprising: receiving new patient data describing at least one of a new cardiac health event determined by a patient implantable medical device (IMD), a record of an arrhythmia treatment applied by the patient IMD, or a programming configuration for the patient IMD. According to such an example, the method may also include outputting a risk-benefit assessment specific to the patient IMD identifying multiple configuration recommendations for the patient IMD and a corresponding risk probability for each of the multiple configuration recommendations based at least in part on application of the new patient data to an Artificial Intelligence model (AI model) trained using a Bayesian network structure to describe cardiac health profiles for a patient population. Subsequent to outputting the risk-benefit assessment specific to the patient IMD, the method includes receiving clinician input selecting one of the multiple configuration recommendations for the patient IMD. Responsive to receiving the clinician input selecting the one of the multiple configuration recommendations for the patient IMD, the method may also include configuring delivery of a therapy via the patient IMD using the one of the multiple configuration recommendations selected according to the clinician input.

In another example, this disclosure describes a non-transitory computer-readable storage medium storing instructions, which when executed, cause processing circuitry to store cardiac data associated with a patient population within a Bayesian network structure. In such an example, the cardiac data describes cardiac health events for the patient population. The instructions, when executed, may also cause the processing circuitry to receive new patient data describing at least one of a new cardiac health event determined by a patient implantable medical device (IMD), a record of an arrhythmia treatment applied by the patient IMD, or a programming configuration for the patient IMD. According to such an example, the instructions, when executed, cause the processing circuitry to output a risk-benefit assessment specific to the patient IMD, wherein the risk-benefit assessment indicates multiple configuration recommendations for the patient IMD and a corresponding risk probability for each of the multiple configuration recommendations based at least in part on application of the new patient data to an Artificial Intelligence model (AI model) trained using the Bayesian network structure to describe cardiac health profiles for a patient population. Subsequent to output of the risk-benefit assessment specific to the patient IMD, the instructions, when executed, may cause the processing circuitry to receive clinician input with a selection of one of the multiple configuration recommendations for the patient IMD. In response to receipt of the clinician input with the selection of one of the multiple configuration recommendations for the patient IMD, the instructions, when executed, may cause the processing circuitry to configure delivery of a therapy via the patient IMD using the one of the multiple configuration recommendations selected according to the clinician input.

In another example, this disclosure describes an apparatus including means for storing cardiac data associated with a patient population within a Bayesian network structure and training the Bayesian network structure to generate per-patient and per-device risk assessments. For instance, the apparatus may include means for receiving new patient data describing at least one of a new cardiac health event determined by a patient implantable medical device (IMD), a record of an arrhythmia treatment applied by the patient IMD, or a programming configuration for the patient IMD. According to such an example, the apparatus may also include means for outputting a risk-benefit assessment specific to the patient IMD identifying multiple configuration recommendations for the patient IMD and a corresponding risk probability for each of the multiple configuration recommendations based at least in part on application of the new patient data to an Artificial Intelligence model (AI model) trained using a Bayesian network structure to describe cardiac health profiles for a patient population. The apparatus may also include, subsequent to outputting the risk-benefit assessment specific to the patient IMD, means for receiving clinician input selecting one of the multiple configuration recommendations for the patient IMD. The apparatus may include means for configuring delivery of a therapy via the patient IMD using the one of the multiple configuration recommendations selected according to the clinician input in response to receiving the clinician input selecting the one of the multiple configuration recommendations for the patient IMD.

The summary is intended to provide an overview of the subject matter described in this disclosure. It is not intended to provide an exclusive or exhaustive explanation of the systems, devices, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates the environment of an example implantable medical device (IMD) in conjunction with a patient, in accordance with aspects of the disclosure.

FIG. 2 illustrates programmable shock vectors for an implantable medical device, in accordance with aspects of the disclosure.

FIG. 3 is a block diagram of a processing system and an external processing system, in accordance with aspects of the disclosure.

FIG. 4 is a functional diagram depicting functions for generating risk projections for an implantable medical device and configuring the implantable medical device, in accordance with aspects of the disclosure.

FIG. 5 illustrates updates to a priori information of a Bayesian network using new patient data, in accordance with aspects of the disclosure.

FIG. 6 is a flow diagram illustrating an example technique for evaluating patient risk associated with IMDs using Bayesian networks, in accordance with aspects of the disclosure. Like reference characters denote like elements throughout the description and figures.

### DETAILED DESCRIPTION

In general, aspects of this disclosure are directed to techniques for generating a risk assessment for output to a clinician utilizing Bayesian Network models trained for quantifying and predicting risk of therapy delivery for various types of Implantable Medical Devices (IMDs) including Implantable Cardioverter Defibrillator (ICD) and Cardiac Resynchronization Therapy Defibrillator (CRT-D) type medical devices. The risk assessment may be generated specifically for the risk to a particular patient, for a particular patient's IMD, for a particular IMD configuration to be applied to the patient's IMD, for a specific type of IMD if implanted into the particular patient, for a specific IMD product if implanted into the particular patient, or some combination thereof. The risk assessment generated may additionally provide as output to a clinician, multiple recommended configurations for a patient IMD with a corresponding risk for each of the recommended configurations. Additional information may be provided as output to the clinician to aid the clinician in selecting a configuration to apply to a patient IMD including, for example, a risk comparison of a given configuration as applied to the particular patient with a patient population having similar or overlapping health characteristics. Responsive to the clinician selecting one of the recommended configurations, the system may responsively configure the patient's IMD with the clinician selected configuration.

Unlike previous techniques which quantify risk for a patient population or some curated patient subpopulation, the system is configured to not only quantify risk for the patient population and/or patient subpopulation, but additionally determine a customized risk calculation for each individual patient and/or each individual device, given various device/patient inputs and/or unique use conditions and observed events for the patient or device evaluated. Modeling results such as patient/device risk assessments and patient/device risk comparisons with a larger population support clinician decision-making with highly-customized patient/device management recommendations unique to the patient in the context of existing clinical trial data and existing field data. In related examples, modeling results provided by the trained Bayesian Network models may be utilized for optimizing IMDs, e.g., determining default values or values of programmable parameters that will be made available for user selection, before product launch. For example, modeling results provided by the trained Bayesian Network models may include a programming configuration projected to result in minimum risk for an entire device/patient population. In other instances, a subset of devices/patients may benefit from a per-device and/or per-patient personalized risk assessment. In such cases, modeling results provided by the trained Bayesian Network models may include IMD configuration values personalized at a per-device and/or per-patient level, based on specific data corresponding to the device and/or patient. Such per-device and/or per-patient configurations may be the same as, or may differ from, an optimized IMD configuration determined for a patient population. Providing risk projections at both the population level and the personalized per-device and/or per-patient level may enable clinicians to make the best possible decision for each patient.

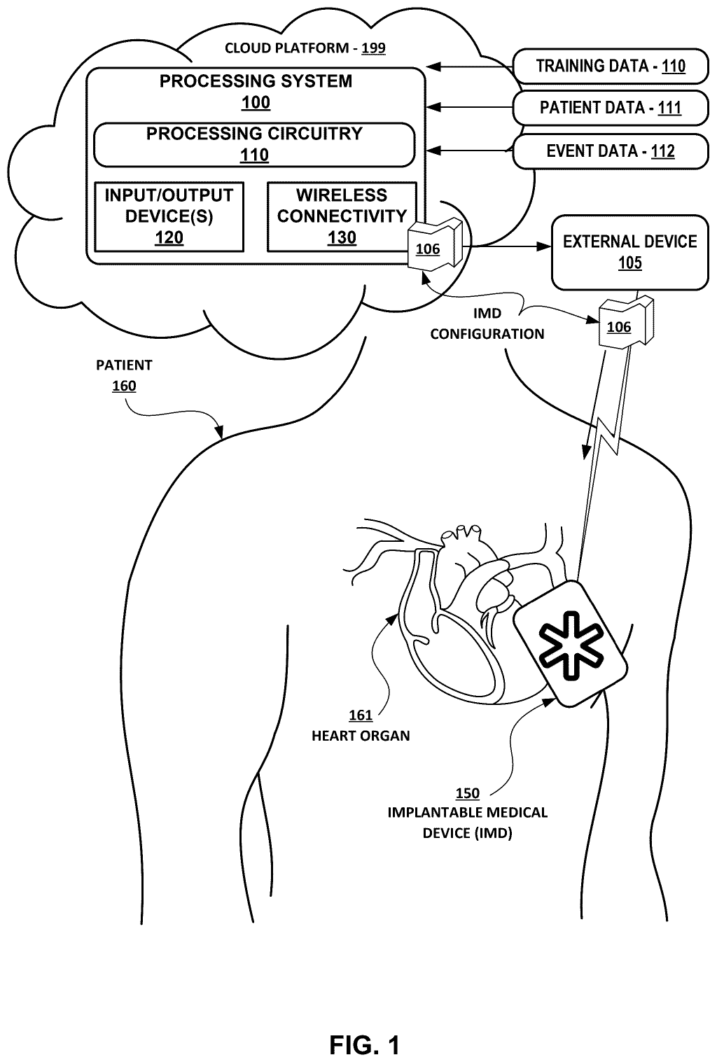

FIG. 1 illustrates the environment of an example implantable medical device (IMD) 150 in conjunction with patient 160, in accordance with aspects of the disclosure.

The example techniques described herein may be used with IMD 150, which may be in wireless communication with at least one of external device 105 and other devices not pictured in FIG. 1. In some examples, IMD 150 may communicate indirectly with processing system 100 through external device 105. In other examples, IMD 150 may communicate with processing system 100 through wireless connectivity 130 without the use of external device 105 (e.g., via a wireless transceiver of processing system 100).

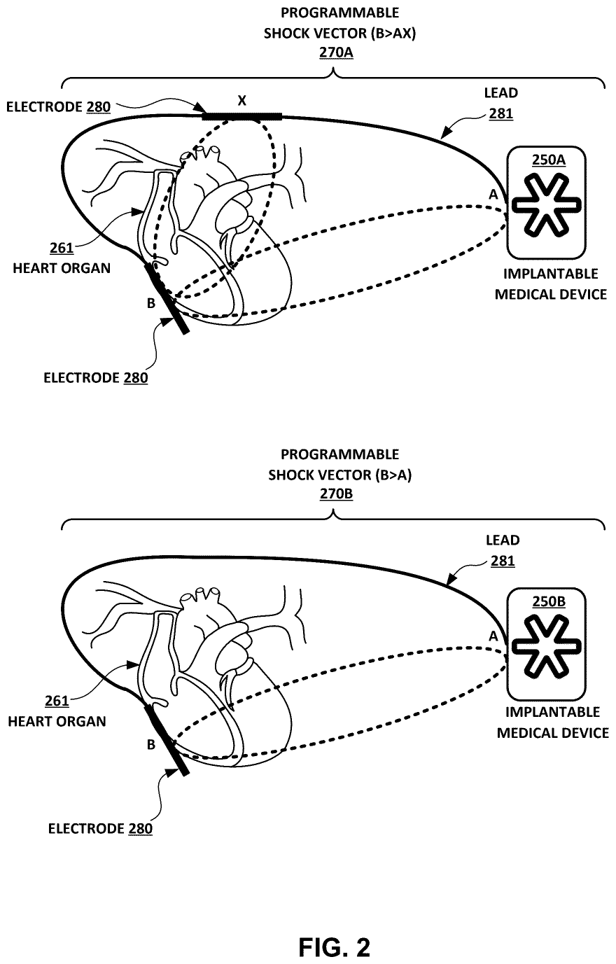

In some examples, IMD 150 is implanted outside of a thoracic cavity of patient 160 (e.g., subcutaneously in the pectoral location illustrated in FIG. 1, a subclavical location, or a lateral location). IMD 150 includes a plurality of electrodes (sec FIG. 2, not shown in FIG. 1), and may be configured to sense cardiac data via the plurality of electrodes, determine cardiac health events via the plurality of electrodes, apply treatment to patient 160 via the plurality of electrodes, or some combination thereof. The electrodes may be disposed on one or more leads and/or a housing of IMD 150, and may be located within heart 161, on heart 161, outside of heart but below a ribcage of patient 160, or subcutaneously.

A variety of types of medical devices sense and collect cardiac data, patient data 111, and/or event data 112, including each of external device 105 and IMD 150. In some examples, cardiac data may include one or more of electrocardiogram (ECG or EKG) signals, electrogram signals, and/or heart sound signals. Some medical devices, including external device 105, that sense and collect cardiac data, patient data 111, and/or event data 112 are non-invasive, e.g., using a plurality of electrodes placed in contact with external portions of the patient, such as at various locations on the skin of the patient. The electrodes used to monitor the cardiac data in these non-invasive processes may be attached to the patient using an adhesive, strap, belt, or vest, as examples, and electrically coupled to a monitoring device, such as an electrocardiogra Holter monitor, or other electronic device. The electrodes are configured to sense electrical signals associated with the electrical activity of heart organ 161 or other cardiac tissue of patient 160, and to provide these sensed electrical signals to the electronic device for further processing and/or display of the electrical signals. For instance, data for a particular patient 160 may be collected by such a non-invasive external device 105 and provided as new patient data 111 to the Bayesian Network models trained for quantifying and predicting risk of therapy delivery. Based on the new patient data collected by the non-invasive external device 105, the trained Bayesian Network models may generate a risk assessment specific to the patient from which the new patient data 111 was collected for IMD 150 to be implanted into patient 160 or for IMD 150 already implanted into patient 160.

The non-invasive devices and methods may be utilized on a temporary basis, for example, to monitor patient 160 during a clinical visit, such as during a doctor's appointment, or for example for a predetermined period of time, for example for one day (twenty-four hours), or for a period of several days.

External devices 105 that may be used to non-invasively sense and monitor cardiac data include wearable devices with electrodes configured to contact the skin of patient 160, such as patches, watches, or necklaces. One example of a wearable physiological monitor configured to sense cardiac data is the SEEQ™ Mobile Cardiac Telemetry System, available from Medtronic, Inc., of Minneapolis, Minnesota. Such external devices 105 may facilitate relatively longer-term monitoring of patients 160 during normal daily activities, and may periodically transmit collected data to processing system 100 over a network service, such as via the Medtronic Carelink™ Network.

External device 105 may be a computing device with a display viewable by a user and an interface for providing input to External device 105 (i.e., a user input mechanism). In some examples, external device 105 may be a notebook computer, tablet computer, workstation, one or more servers, cellular phone, smartphone, personal digital assistant, or another computing device that may run an application that enables the computing device to interact with IMD 150. External device 105 is configured to communicate with IMD 150 and, optionally, another computing device such as processing system 100, via wireless communication. External device 105, for example, may communicate with IMD 150, processing system 100, or both, via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies).

Processing system 100 includes processing circuitry 110 configurable to execute instructions, operate functional components, and perform other operations described herein in accordance with one or more aspects of the disclosure. Processing system 100 may operate within cloud platform 199, such as an on-demand service provider accessible to subscribers and/or users via a public Internet. For instance, processing system 100 may operate within cloud platform 199 and communicate with a patient smartphone device operating as external device 105 over a public Internet or communicate with a clinician's computing device in a healthcare setting over a public Internet. Processing system 100 includes input/output devices 120 via which to receive and send information and wireless connectivity 130 via which to communicate with other computing devices, such as external device 105, indirectly with IMD 150 via external device 105, and/or directly with IMD 150 via wireless connectivity 130.

Processing system 100 may obtain information via input/output devices 120 for processing such as training data 110, patient data 111, and event data 112. Training data 110 may include, for example, a priori information, such as existing clinical data for a patient population and existing field data for a patient population. Patient data 111 may include, for example, new patient information for a particular patient obtained from a clinician, obtained from an Electronic Medical Record (EMR) system having information about a particular patient, from external device 105 having collected information about a patient, from IMD 150 implanted within patient 160, from a clinician user interface having obtained input from a clinician about patient 160, etc. Event data 112 may include health event information determined by external device 105 having collected event data 112 associated with patient 160, from IMD 150 implanted within patient 160 and having collected event data 112 associated with patient 160, from a clinician user interface having obtained input indicating event data 112 from a clinician about patient 160, etc. For instance, event data 112 may include a record of detection and treatment of an event, such as an arrhythmia or other cardiac health event.

In some examples, cardiac data may include health event data 112 associated with patient 160, new patient data 111 for patient 160, cardiac data sensed by IMD 150, or some combination thereof. In some examples, processing circuitry, such as processing circuitry 110 of processing system, processing circuitry of IMD 150, processing circuitry of external device 105, and/or processing circuitry of a cloud computing device, determine, collect, send/receive, and apply collected patient data 111 and/or event data 112 to a Bayesian network model trained to quantify and predict the risk of therapy delivery to IMD 150. In some examples, the Bayesian network model may assign a cardiac event probability percentage to one or more cardiac data samples based on collected patient data 111 and/or event data 112. Processing circuitry 110 of processing system 100 may generate a risk assessment for patient 160, for a particular patient 160 implanted with IMD 150 based on new patient data 111 specific to the patient 160 and existing clinical and field data for a patient population represented within training data 110. In some examples, processing circuitry 110 may apply a model, such as a machine learning model or an Artificial Intelligence (AI) model, to generate a risk assessment for each one of multiple possible IMD configurations 160 customized specifically for a particular patient 160 based on collected patient data 111 and heal event data 112 associated with patient 160. Such models may utilize a Bayesian network structure or include a Bayesian network model trained to quantify and predict the risk of therapy delivery by IMD 150.

Examples of event data 112 about patient 160 may include, for example, determined arrhythmia events, such as determined Atrial Fibrillation (AF) events, other episodes of irregular or rapid heartbeats of heart organ 161 originating from the atria, bradycardia events including episodes of abnormally slow heart rate of heart organ 161, tachycardia events including episodes of abnormally fast heart rate, atrial flutter events including episodes of rapid but regular atrial heart rhythm, ventricular arrhythmias including abnormal heart rhythms originating in the ventricles, such as ventricular tachycardia or fibrillation, heart rate variability events including fluctuations in heart rate recorded over time, syncope (fainting) events which may be potentially caused by arrhythmias, postural changes including detection of changes in heart rate and rhythm associated with standing up or lying down, physical activity induced heart rate changes in response to exercise or physical exertion. Such event data 112 will be uniquely peculiar to patient 160 from which event data 112 is collected. However, patient 160 specific event data 112 may be analyzed in the context of larger information domains associated with a larger set of patients, especially when narrowed to a subset of a patient population having similar or overlapping health characteristics as patient 160 based on patient data 111 (e.g., similar age, gender, risk factors, comorbidities, heart condition diagnosis, etc.).

Examples of patient data 111 about patient 160 may include, for example, patient age, patient gender, patient income, patient education, patient health history, patient family health history, and other demographic information for patient 160. Sub-categories of event data and patient data 111 about patient 160 may include device data and therapy history data. Each of device data and therapy history data may similarly be provided as inputs to the Bayesian network model to project risk on a per-patient/per device basis. Device data may include data detected and/or collected by processing system 100 indicating physician inputs, programming configuration, device service life, etc. Therapy history data may include therapy history for IMD 150 and/or patient 160.

In some examples, external device 105 may be or additionally include a wearable computing device. A wearable computing device may include electrodes and other sensors to sense physiological signals of patient 160, and may collect and store physiological data and detect episodes based on such signals. Wearable computing devices may be incorporated into the apparel of patient 160, such as within clothing, shoes, eyeglasses, a watch or wristband, a hat, etc. In some examples, a wearable device may be a smartwatch or other accessory or peripheral for external device 105, for example when external device 105 is a smartphone or tablet.

External device 105 may be used to provision, download, install, or otherwise configure IMD 150 with IMD configuration 106. IMD configuration 106 is utilized to configure operational parameters for IMD 150. External device 105 may be used to retrieve data from IMD 150. The retrieved data may include health event data 112 associated with patient 160, new patient data 111 for patient 160, cardiac data sensed by IMD 150, or some combination thereof. In some examples, cardiac data may include an ECG signal. In some examples, a set of cardiac data may include a plurality of cardiac data samples. In other examples, a set of cardiac data includes physiological indicators for patient 160.

External device 105 may also retrieve set(s) of patient data 111, event data 112, and cardiac data recorded by IMD 150, e.g., according to a schedule, due to IMD 150 determining that an acute cardiac event, such as an arrhythmia, occurred, or in response to a request to record the segment from patient 160 or another user. One or more remote computing devices, such as processing system 100, a clinician user interface at a clinician computing device, or an internet connected cloud platform may interact with IMD 150 in a manner similar to external device 105, e.g., to program IMD 150 and/or retrieve data from IMD 150, via a network.

Processing circuitry 110 of processing system 100, processing circuitry of external device 105, processing circuitry of IMD 150, and/or of one or more other computing devices, may be configured to perform the example techniques of this disclosure for causing a user interface to display a set of cardiac data, patient data 111, and/or event data 112, in which such data may be associated with a cardiac related health event of patient 160, receiving, from a user interface, a user selection of cardiac data, patient data 111, and/or event data 112 corresponding to a cardiac health event. Any or all of cardiac data, patient data 111, and/or event data 112 may be obtained by processing system 100 and applied to Bayesian Network models trained for quantifying and predicting risk of therapy delivery to generate a patient specific and/or IMD 150 specific risk assessment for patient 160, in accordance with the techniques described herein.

Processing circuitry 110 may include fixed function circuitry and/or programmable processing circuitry. Processing circuitry 110 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 110 may include multiple components, such as any combination of one or more microprocessors, one or more controllers, one or more DSPs, one or more ASICs, or one or more FPGAs, as well as other discrete or integrated logic circuitry. The functions attributed to processing circuitry 110 herein may be embodied as software, firmware, hardware or any combination thereof.

In some examples, processing circuitry 110 may store a plurality of sets of the cardiac data, such as digitized ECG signal and/or heart sound beat signal in a storage device. Processing circuitry 110 of processing system 100, processing circuitry of IMD 150, and/or processing circuitry of external device 105 that retrieves data from IMD 150, may analyze the cardiac data to determine and assign a cardiac event probability percentage to one or more cardiac data samples of a set of the cardiac data that a respective cardiac data sample is associated with the cardiac event. In some examples, processing circuitry 110 of IMD 150, and/or processing circuitry of another device that retrieves data from IMD 150, may analyze the cardiac data to determine an acute cardiac event occurred.

Wireless connectivity 130 may include communication circuitry, such as a transceiver. Communication circuitry may include any suitable hardware, firmware, software or any combination thereof for communicating with another device, such as external device 105, another networked computing device, or another IMD 150 or sensor. Under the control of processing circuitry 110, communication circuitry may receive downlink telemetry from, as well as send uplink telemetry to external device 105 or another device with the aid of an internal or external antenna. In addition, processing circuitry 110 may communicate with a networked computing device via an external device (e.g., external device 105) and a computer network, such as the Medtronic CareLink™ Network. Wireless connectivity 130 may utilize an antenna and communication circuitry configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, processing system 100, external device 105, and/or IMD 150 includes a storage device having computer-readable instructions that, when executed by processing circuitry 110, cause IMD 150 and processing circuitry 110 to perform various functions attributed to IMD 150 and processing circuitry 110 herein. A storage device may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media. Such a storage device may store, as examples, programmed values for one or more operational parameters of IMD 150 and/or data collected by IMD 150 for transmission to another device using communication circuitry. Data stored by a storage device and transmitted by communication circuitry to one or more other devices may include one or more sets of cardiac data, digitized ECG signals, and/or digitized heart sound beat signals, event data 112, patient data 111, as examples.

FIG. 2 illustrates programmable shock vectors for implantable medical device (IMD) 250A, 250B, in accordance with aspects of the disclosure. FIG. 2 shows programmable shock vector 270A (B to AX) and programmable shock vector 270B (B to A) configured for IMD 250A and 250B, respectively. Leads 281 are depicted as originating from IMDs 250A-B and electrically connecting IMDs 250A-B with heart organ 261. For IMD 250A, leads 281 include electrodes 280 positioned at positions (B,X) of heart organ 261, with IMD 250A being located at position (A). For IMD 250B, leads 281 include electrodes 280 positioned at position (B) of heart organ 261, with IMD 250A being located at position (A). In addition to electrodes 280, a housing of IMDs 250 may be conductive, or otherwise include an electrode, and thus form one of the poles of the shock vectors illustrated in FIG. 2. In other examples, one or more electrodes 280 may be positioned within heart organ 261 via an intravascular type lead 281. According to such examples, lead 281 may be formed from a thin, insulated wire that carries electrical impulses from IMD 250A-B to heart organ 261. An intravascular lead 281 may have the wire positioned inside a blood vessel, directed into heart organ 261, and contacting heart tissue to deliver electrical signals, pulses, shocks, etc. to heart organ 261.

FIG. 2 shows how programmable shocking vectors 270A-B may be applied to heart organ 261 from position B to AX for IMD 250A and from position B to A for IMD 250B. A nominal programmable shocking vector from B to AX means energy is delivered from electrode at position B to each of positions A and X in the first phase of programmable shock vector 270A and subsequently from position AX to B in a second phase of programmable shock vector 270A during a biphasic high voltage shock. Programmable shock vector 270B depicts B to A, meaning energy is delivered from electrode at position B to position A in the first phase of programmable shock vector 270B and subsequently from position A to B in a second phase of programmable shock vector 270B.

IMD 250A-B may be configured to deliver up to 6 high voltage shocks during an episode per the system design and configuration of IMD 250A with different permissible programmable shock vectors 270A-B. Consequently, there are 2\hat{ }6 =64 programmable pathway configurations for IMD 250A. For example, programmed pathway “BBBBAA” in this document means that the first four out of six shocks are programmed to be B to AX in the first phase and the last two shocks are programmed to be AX to B in the first phase. The chance of each shock being delivered depends on efficacy. As most arrhythmia episodes will be terminated by the first shock, most patients only experience one shock during an episode. However, subsequent shocks may be conditionally delivered dependent on an outcome of a prior shock. Stated differently, IMD 250A-B will not shock a patient a second time when a first shock delivers an effective therapy sufficient to terminate an arrhythmia episode. A second, third, fourth, fifth, and sixth shock may, however, be delivered to heart organ 261 by IMD 250A-B if prior shocks for a determined arrhythmia episode fail to terminate the arrhythmia.

For instance, IMD 250A-B may be configured to sense and monitor cardiac data and conditionally treat determined cardiac events satisfying various thresholds. Electrodes 280 may be used by IMD 250A-B to sense cardiac data. Electrodes 280 may be integrated with a housing of IMD 250A-B and/or coupled with cardiac tissue of heart organ 261 back to IMD 250A-B via one or more elongated leads 281 electrically interfacing IMD 250A-B with electrodes 280. Example IMD 250A-B that monitor cardiac data include pacemakers and implantable cardioverter-defibrillators, which may be coupled to intravascular or extravascular leads 281, as well as pacemakers with housings configured for implantation within heart organ 261, which may be leadless, thus having electrodes 280 positioned at IMD 250A-B. An example of pacemaker configured for intracardiac implantation is the Micra™ Transcatheter Pacing System, available from Medtronic, Inc. Some IMDs that do not provide therapy, e.g., implantable patient monitors, sense cardiac data. One example of such an IMD is the Reveal LINQ™ and LINQ II™ Insertable Cardiac Monitors (ICMs), available from Medtronic, Inc, which may be inserted subcutaneously. Such IMDs may facilitate relatively longer-term monitoring of patients during normal daily activities, and may periodically transmit collected data to a network service via external device 105 (see FIG. 1). For instance, data may be obtained from IMD 250A-B via the Medtronic Carelink™ Network. In some examples, such IMDs 250A-B may transmit sensed cardiac data to an external computing device such as processing system 100 (see FIG. 1) directly or indirectly using an external device 105.

Any medical device configured to sense cardiac data, such as via implanted or external electrodes 280, including the examples identified herein, may sense a plurality of sets of cardiac data. In some examples, a medical device, such as an IMD, may include a memory and store the plurality of sets of cardiac in the memory. In some examples, an external computing device may store the plurality of sets of cardiac data.

In some examples, a patient IMD 250A-B may be configured to deliver general therapy, deliver High Voltage (HV) therapy including defibrillation, deliver multiple conditional successive HV therapies, or deliver non-HV therapy such as ATP or other pacing pulses.

One type of IMDs 250A-B includes ICDs. ICDs treat abnormal heart rhythms (ventricular arrhythmias) such as ventricular tachycardia and ventricular fibrillation. These life-threatening rhythms can cause sudden cardiac arrest (SCA), which is lethal if not treated. ICDs treat arrhythmias by delivering high voltage shocks (up to 800V), when there is an arrhythmia episode determined by the ICD. For example, an ICD may be configured to deliver a sequence of shocks (up to 6 shocks by way of example) per episode. With treatment, 98% of patients will survive an otherwise lethal arrhythmia when treated with defibrillation. In some examples, ICDs may treat arrhythmias by delivering therapies according to a sequence that includes ATP in addition to shocks, e.g., before shocks.

Another type of IMD 250A-B is a Cardiac Resynchronization Therapy (CRT) type medical device. CRTs treat heart failure by delivering biventricular pacing to correct electrical dyssynchrony. Electrical dyssynchrony refers to a condition where the electrical impulses that coordinate the heart's pumping action are disrupted or delayed. This may occur due to heart disease, myocardial infarction (e.g., a heart attack), or cardiomyopathy (e.g., disease of the heart muscle). When the electrical signals of the patient's heart are not synchronized properly, the chambers (ventricles) of the heart may not contract together efficiently. With a CRT type medical device implanted, both ventricles are paced. The result is a more coordinated contraction and increased cardiac output.

Another type of IMD is a Cardiac Resynchronization Therapy Defibrillator (CRT-D) which provides cardiac resynchronization therapy with pacing and an ICD, for patients diagnosed with heart failure who also have a risk of sudden cardiac death.

In some examples, processing circuitry 110 of a processing system 100 (see FIG. 1) may receive input, such as from a clinician user interface, indicating a clinician's selection of one of the multiple recommended IMD configurations 106 (see FIG. 1) generated by a trained Bayesian network model. Processing circuitry may cause the user interface, such as a clinician user interface of an external computing device 100, to indicate additional detail for the selected IMD configuration 106, such as a comparison of risk to patient 160 (see FIG. 1) compared with a larger patient population based on existing clinical and field data. Customized comparisons may be generated based on further input obtained by the clinician user interface, such as a risk to patient 160 compared with a patient sub-population selected based on overlapping health characteristics to patient 160 according to patient data 111, such as similar age, gender, risk factors, comorbidities, IMD 250A-B type, IMD 250A-B product, and so forth. In some examples, responsive to a clinician indicating a selection of a recommended configuration output by the Bayesian network model, processing circuitry may also be configured to configure IMD 250A-B using a clinician selected IMD configuration 160.

In some examples, configuring cardiac event probability thresholds on a patient-by-patient basis may generate an improved display on a user interface that may reduce an amount of time that a clinician has to spend identifying an appropriate therapy prescription and corresponding IMD configuration for a particular patient.

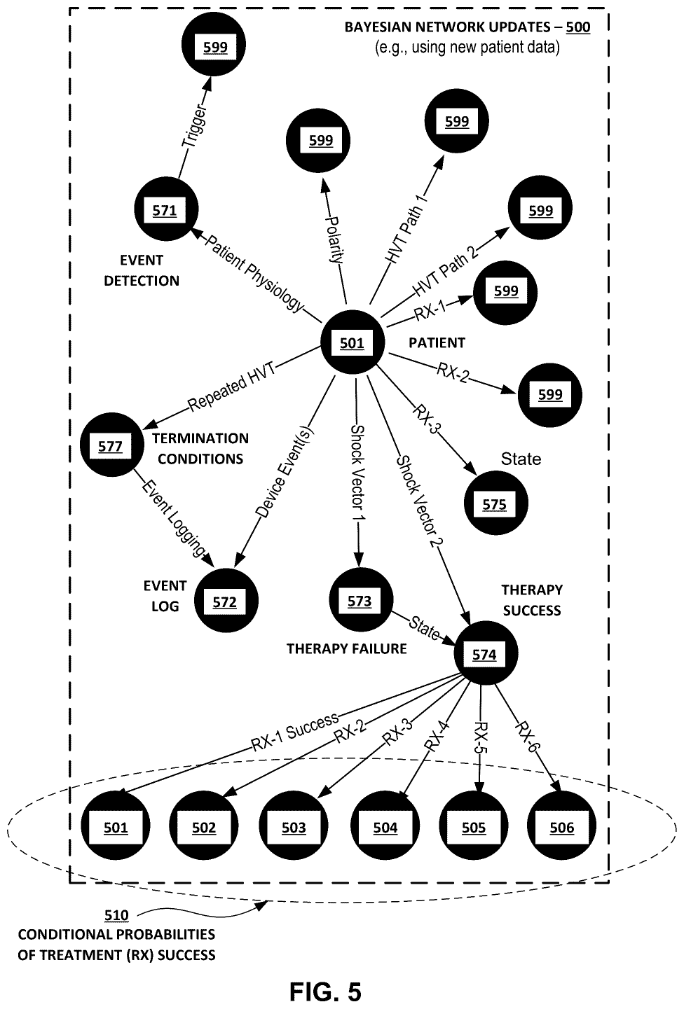

Trained Bayesian network models may provide projected risk of therapy delivery for the ICDs and CRT-Ds device population, for various subpopulations, and for each individual IMD 250A-B device for each of multiple different programming configurations, where risk for some subpopulations cannot be quantified directly from experimental or field data due to data/sample size limitations. Inputs to trained Bayesian network models may include system design (e.g., programmable shock vectors 270A-B specifying a sequence of six HV therapies during an episode), parametric survival analysis results depending on device data, therapy efficacy data, use condition distributions, and other conditional probabilities 510 (see FIG. 5).

Based on model and simulation results, programming recommendations can be provided in the form of recommended IMD configurations 106 (see FIG. 1). Risk can be assessed for various subpopulations for each recommended IMD configuration 106 and additionally compared with a particular patient via output to a clinician display interface. Customized risk-benefit assessment can be made for individual devices/patients at various situations per the input data, directly impacting patients in a positive way and providing clinicians with improved data upon which to make patient care related decisions.

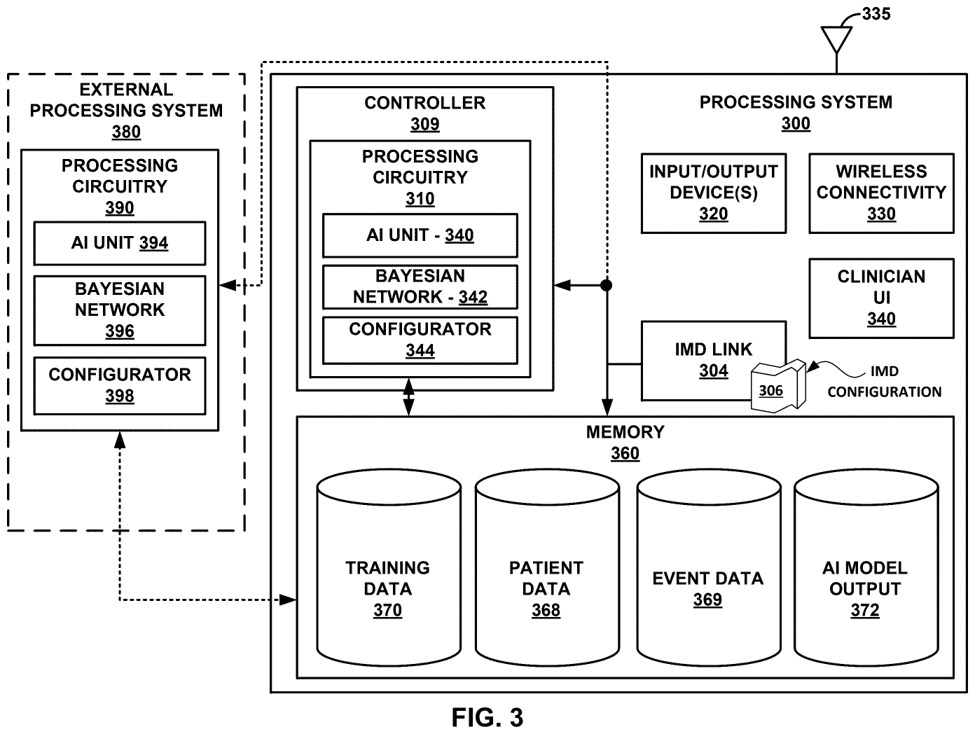

FIG. 3 is a block diagram of processing system 300 and external processing system 380, in accordance with aspects of the disclosure. The functions of FIG. 3 may be implemented using processing system 100 and IMD 150 of FIG. 1, and IMDs 250A-B of FIG. 2, or some combination thereof.

Processing system 300 may be used in conjunction with an apparatus, such as IMD 150 of FIG. 1, external device 105 of FIG. 1, and IMD 250 of FIG. 2. Processing system 300 may include IMD link 304 for exchanging information with an IMD including sending IMD configuration to an IMD, controller 309, input/output device(s) 320, wireless connectivity component 330, clinician user interface (UI) 340, and memory 360.

Wireless connectivity component 330 may include subcomponents, for example, for third generation (3G) connectivity, fourth generation (4G) connectivity (e.g., 4G Long Term Evolution (LTE)), fifth generation (5G) connectivity (e.g., 5G or New Radio (NR)), Wi-Fi connectivity, Bluetooth connectivity, and other wireless data transmission standards. Wireless connectivity component 330 is further connected to one or more antennas 335.

Processing system 300 may also include one or more input and/or output devices 320, such as screens, touch-sensitive surfaces (including touch-sensitive displays), physical buttons, speakers, microphones, and the like. Input/output device(s) 320 (e.g., which may include an I/O controller) may manage input and output signals for processing system 300. In some cases, input/output device(s) 320 may represent a physical connection or port to an external peripheral. In some cases, input/output device(s) 320 may utilize an operating system. In other cases, input/output device(s) 320 may represent or interact with a modem, a keyboard, a mouse, a touchscreen, or a similar device. In some cases, input/output device(s) 320 may be implemented as part of a processor (e.g., a processor of processing circuitry 310). In some cases, a user may interact with a device via input/output device(s) 320 or via hardware components controlled by input/output device(s) 320.

Controller 309 may be configured to control operation of processing system 300 (e.g., including generating risk assessments and configuring an IMD via IMD link 304). For example, controller 309 may control UI interactions with a clinician interface, control the generation and output of recommended IMD configurations, control providing risk comparisons to a clinician UI 340, etc. Controller 309 may include one or more processors, e.g., processing circuitry 310. Processing circuitry 310 may include one or more central processing units (CPUs), such as single-core or multi-core CPUs, graphics processing units (GPUs), digital signal processor (DSPs), application specific integrated circuits (ASICs), field programmable gate arrays (FPGAs), neural processing unit (NPUs), multimedia processing units, and/or the like.

Instructions applied by processing circuitry 310 may be loaded, for example, from memory 360 and may cause processing circuitry 310 to perform the operations attributed to processor(s) in this disclosure. In some examples, one or more of processing circuitry 310 may be based on an Advanced Reduced Instruction Set Computer (RISC) Machine (ARM) or a RISC five (RISC-V) instruction set.

An NPU is generally a specialized circuit configured for implementing control and arithmetic logic for executing machine learning algorithms, such as algorithms for processing artificial neural networks (ANNs), deep neural networks (DNNs), random forests (RFs), kernel methods, and the like. An NPU may sometimes alternatively be referred to as a neural signal processor (NSP), a tensor processing unit (TPU), a neural network processor (NNP), an intelligence processing unit (IPU), or a vision processing unit (VPU).

Processing circuitry 310 may also include one or more sensor processing units associated with IMD link 304. For example, processing circuitry 310 may include one or more image signal processors to process information obtained via IMD link 304.

Processing system 300 also includes memory 360, which is representative of one or more static and/or dynamic memories, such as a dynamic random-access memory, a flash-based static memory, and the like. In this example, memory 360 includes computer-executable components, which may be applied by one or more of the aforementioned components of processing system 300.

Examples of memory 360 include random access memory (RAM), read-only memory (ROM), electrically erasable programmable ROM (EEPROM), compact disk ROM (CD-ROM), or another kind of hard disk. Examples of memory 360 include solid state memory and a hard disk drive. In some examples, memory 360 is used to store computer-readable, computer-executable software including instructions that, when applied, cause a processor to perform various functions described herein. In some cases, memory 360 contains, among other things, a basic input/output system (BIOS) which controls basic hardware or software operation such as the interaction with peripheral components or devices. In some cases, a memory controller operates memory cells. For example, the memory controller may include a row decoder, column decoder, or both. In some cases, memory cells within memory 360 store information in the form of a logical state.

Processing system 300 may be configured to perform techniques for obtaining sensor inputs via IMD link 304 and clinician UI 340, including new patient data 368, health event data 369 and clinician inputs, selections, and requests for additional information associated with a generated risk assessment. In certain examples, processing system 300 is configured to process sensor inputs and clinician UI 340 inputs obtained utilizing AI unit 340 having been trained on Bayesian Network 396 of external processing system 380 for quantifying and predicting risk of therapy delivery to generate a patient specific and/or IMD specific risk assessment for patient. In some examples, AI unit 340 may be trained on Bayesian Network 342 of controller 309. For example, AI unit 340 may be trained on a local Bayesian Network 342 or AI unit 340 may be trained on a remote Bayesian Network 396 within external processing system 380. For instance, AI unit 340 of controller 309 may communicate with a cloud system implemented by external processing system 380 to train AI unit 340 utilizing Bayesian Network 396. AI unit 394 may similarly be located within external processing system 380 and may be trained on Bayesian Network 396 for quantifying and predicting risk of therapy delivery. AI unit 340, 394 may generate multiple IMD configurations 306 utilizing a Bayesian network structure of Bayesian Network 342, 396 trained on existing clinical data and existing field data to represent a patient population. Bayesian Network 342, 396 may be iteratively updated to incorporate new patient data 368 and health events 369 for patient 160 (see FIG. 1). Based on input from clinician UI 340, an IMD configuration 306 may be selected to be installed or configured into IMD 150 for patient 160. Configurator 344, 398 may apply the clinician selected IMD configuration 306 to IMD 150 for patient 160.

In some examples, processing circuitry 310 may be configured to train one or more machine learning models such as encoders, decoders, positional encoding models, or any combination thereof applied by AI unit 340, 394 using training data 370. For example, training data 370 may include existing clinical data and existing field data to represent a patient population. Training data 370 may allow processing circuitry 310 to train an encoder to generate features that accurately represent a patient population.

As discussed above, aspects of the techniques of this disclosure may be performed by external processing system 380. That is, encoding input data, transforming features into Bayesian network 396 may be performed by a processing system that does not include the various components shown for processing system 300 such as IMD link 304 and clinician UI 340. Such a process may be referred to as “offline” data processing, where the output is determined from sensor inputs information obtained from processing system 300 and processed by external processing system 380. In some examples, external processing system 380 updates an AI unit 394 to generate per-patient and per IMD device risk assessments based on new patient data 368 and/or new event data 369 associated with a patient obtained from processing system 300, and configurator 398 provides recommended IMD configurations 306 back to processing system 300. Processing system 300 may subsequently present the multiple recommended configurations 306 to clinician UI 340 and responsive to one IMD configuration 306 being selected based on clinician UI 340 input, configurator 344 may configure a patient IMD with the selected IMD configuration 306 using IMD link 304. Similarly, external processing system 380 may process information obtained from processing system 300 at inference time using AI unit 394 and send output to processing system 300, for instance, providing risk assessments and/or recommended IMD configurations 306 to processing system 300 for output via clinician UI 340.

AI model inference is the process of applying a previously trained AI model to input data, such as sensor input, new patient data 368, new event data 369, etc., to make predictions, decisions, or generate output for other downstream tasks. AI model inference uses the learned parameters of the trained AI model to interpret new and previously unseen data and generate meaningful outputs.

External processing system 380 may include processing circuitry 390, which may be any of the types of processors described above for processing circuitry 310. Processing circuitry 390 may include AI unit 394 configured to perform the same processes as AI unit 340. Processing circuitry 390 may acquire sensor input from an IMD, acquire patient data 368, and/or acquire event data 369, via processing or retrieve such information from memory 360. Though not shown, external processing system 380 may also include a memory that may be configured to store sensor inputs, patient data 368, event data 369, and provide model outputs, including AI model output 372, among other data that may be used in data processing. AI unit 394 may be configured to perform any of the techniques described as being performed by AI unit 340. Bayesian network 396 may be configured to perform any of the techniques described as being performed by Bayesian network 342 including quantifying and predicting risk of therapy delivery.

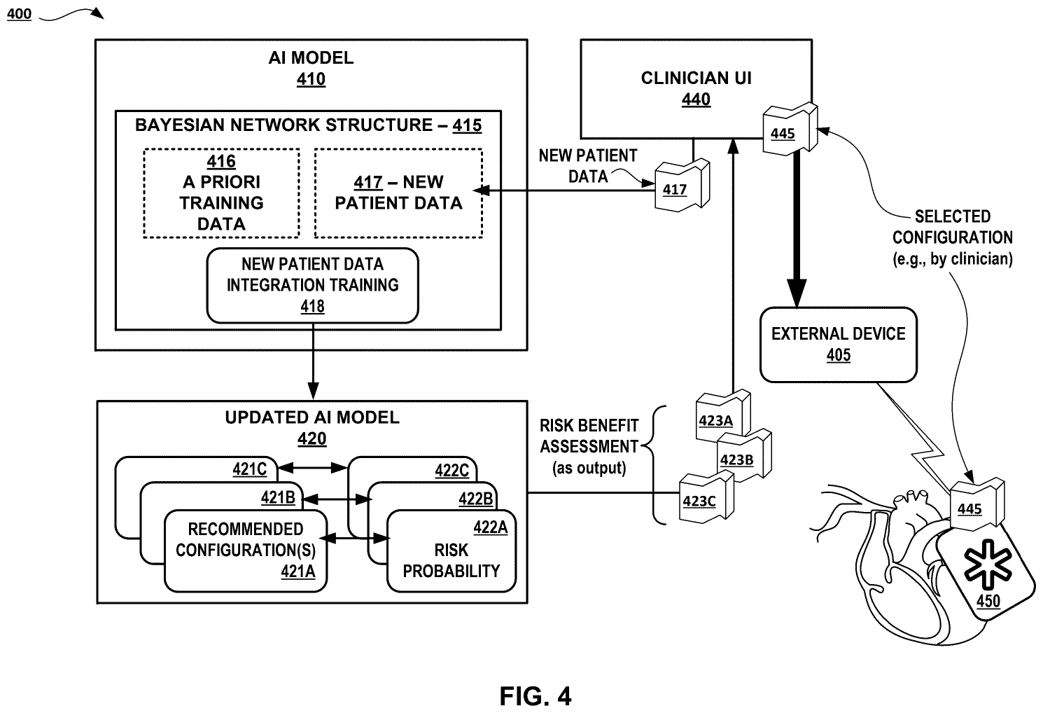

FIG. 4 is a functional diagram 400 depicting functions for generating risk projections for implantable medical device 450 and configuring implantable medical device 450, in accordance with aspects of the disclosure. The functions of FIG. 4 may be implemented using processing system 100 and IMD 150 of FIG. 1, IMDs 250A-B of FIG. 2, processing system 300 of FIG. 3, external processing system 380 of FIG. 3, or some combination thereof.

As shown here, AI model 420 includes a Bayesian network structure 415 having a priori training data 416 and new patient data 417. Bayesian network structure 415 may be trained on a priori training data 416 including existing clinical data and existing field data to represent a patient population. AI model 410 may iteratively update Bayesian network structure 415 using new patient data integration training 418 to incorporate new patient data 417 into Bayesian network structure 415. New patient data 417 may be obtained from clinician UI 440.

Updating Bayesian network structure 415 provides updated AI model 420 having Bayesian network structure 415 representing both a larger patient population based on a priori training data 416 and new patient data 417 unique to a particular patient for which new patient data 417 was originated.

Updated AI model 420 may generate recommended configurations 421A, 421B, and 421C and corresponding risk probabilities 422A, 422B, and 422C. Any number of recommended configurations 421A-C may be generated. Notably, each recommended configuration 421A-C may be associated with corresponding risk probability 422A-C specific to one recommended configuration 421A-C. In such a way, each recommended configuration 421A-C may have a different and distinct risk probability 422A-C.

Updated AI model 420 may provide recommended configurations 421A-C and corresponding risk probabilities 422A-C as output via risk benefit assessments 423A, 423B, 423C. Risk benefit assessments 423A-C are provided as output and for display to clinician UI 440.

Clinician input at clinician UI 440 may indicate a selected configuration 445, responsive to which, selected configuration 445 may be sent to external device 405. As shown here, external device 405 installs selected configuration 445 into IMD 450 for patient 160 (sec FIG. 1).

Bayesian network structure 415 may be learned from existing clinical trial data and existing field data. AI model 410 may be updated when a programming configuration 445 is selected at clinician UI 440 and/or when one or more events associated with patient 160 (see FIG. 1) are observed or based on new patient data for patient 160 is obtained. With results from AI model 410 utilizing Bayesian network structure 415, physicians are able to quantify risk for multiple candidate pathway configurations for a patient and compare that risk with other types of risk based on risk assessments 423A-C provided to a clinician, thus enabling the clinician select their preferred solution which may be unique to one specific patient. For some patients, there may be a balance between minimizing risk related to IMD 450 device events and other risks associated with undesirable side effects.

In some examples, Bayesian network structure 415 may be trained based on a given probability of an event, depending on device type/polarity etc. In some examples, Bayesian network structure 415 may be trained based on distribution of programmed polarities based on field data. In some examples, Bayesian network structure 415 may be trained based on intermittency of events based on determined pathways. In some examples, Bayesian network structure 415 may be trained based therapy efficacy based on a quantity of shocks delivered during a single health event. Based on the training of Bayesian network structure 415, AI model 410 may generate, utilizing Bayesian network structure 415, a probability that IMD 450 device is unable to provide successful therapy based on different pathway configurations given the permissible sequences of 6 shocks.

According to certain examples, AI model 410 utilizing Bayesian network structure 445 accommodates IMD 450 system enabling delivery of up to 6 shocks for a determined episode to terminate arrhythmia. AI model 410 may determine prior probability and conditional probability of nodes in the Bayesian network structure 415 according to any of survival analysis results, therapy efficacy data, historical events data, use condition data etc. With each known programmed pathway configuration (e.g., AAAAAA, BBBBBB, ABABAB programmable shock vectors for IMD 450), the efficacy probability at the end of the episode consisting of up to six shocks is calculated by AI model 410 per Bayesian network structure 415. With each known observed event information, the posterior probability of each node in Bayesian network structure 415 is calculated by AI model 410 according to the prior probabilities and the conditional probabilities. Risk probability 422A-C is provided and/or visualized for each patient subpopulation and each individual IMD 450 device given the programmed pathway configuration, device/manufacturing type, implant duration, observed events for a given patient, etc. Risk comparison may be analyzed by clinicians based on risk benefit assessments 423A-C provided as output to support clinician decision making in each unique scenario. In other examples, risk may be assessed to guide new design and pre-market product launch for IMD 450 at design time, during prototyping, testing, etc.

FIG. 5 illustrates updates to a priori information 501 of a Bayesian network using new patient data, in accordance with aspects of the disclosure. For instance, FIG. 5 depicts Bayesian network updates 500 being applied to a previously trained Bayesian network structure using new patient data. As described above, the Bayesian network model may be trained to estimate therapy efficacy by up to 6 shocks per episode for a particular patient IMD utilizing the Bayesian network updates 500.

AI model 410 (see FIG. 4) may recursively update the Bayesian network structure using the new patient data by updating one or more conditional probability tables of the Bayesian network structure using the new patient data. AI model 410 may subsequently output the risk-benefit assessment specific to the patient IMD using the one or more conditional probability tables updated using the new patient data. In such an example, the Bayesian network structure may describe the cardiac health profiles for the patient population based on existing clinical trial data and existing field data and the Bayesian network updates 500 describe health events and new patient data unique to a particular patient which was not represented by a priori training data.

As depicted by FIG. 5, the Bayesian network structure includes a priori information 501 near the center, linked to various nodes 599 specifying parameters, links, and relationships, such as polarity, HVT path 1, HVT path 2, prescription 1 (RX-1) for a patient, RX-2, RX-3, shock vector 1, shock vector 2, etc. Additional nodes of Bayesian network structure depict state 575 information, therapy failure 573, therapy success 574, termination conditions 577, event logging for an event log 572, event detection 571 and triggers, etc. Various conditional probabilities of treatment (RX) success 510 are depicted for each of the 6 permissible conditional shocks, including probability of RX-1 success at conditional probability node 501, probability of RX-2 success at conditional probability node 502, probability of RX-3 success at conditional probability node 503, probability of RX-4 success at conditional probability node 504, probability of RX-5 success at conditional probability node 505, and probability of RX-6 success at conditional probability node 506.

Elements in the Bayesian network structure may include, but are not limited to the following examples: In an episode, there are up to 6 shocks in a therapy Efficacy of each shock (probably of therapy success given a shock) is different and is dependent on the shock number and whether there is an event in the current shock for a regular shock without an event, the probability of therapy failure is dependent on shock number. For example, the 1st shock has the highest probability of therapy success compared to shock numbers 2 to 6. For a shock with an event, the probability of therapy failure is calculated based on existing field data. An event can happen on any shock. Probability of an event is dependent on the device type A>B vs. B>A path (or AX>B vs. B>AX path) as described above with reference to FIG. 2. Probability of an event for the current shock depends on whether an event has already occurred and, if an event has occurred, the path of the first event. If an event has not yet occurred, then use P(event|A>B, no event in previous shocks) & P(event|B>A, no event in previous shocks) rates (use the first event rates from survival and competing risk analysis). If the first event was “B-A”−use P(event|A>B, event in previous shocks, first event path=B>A) & P(event|A>B, event in previous shocks, first event path=B>A) rates. If the first event was “A-B”−use P(event|A>B, event in previous shocks, first event path=A>B) & P(event|B>A, event in previous shocks, first event path=A>B) rates.

Consider the following application specific examples. If a certain type of device is programmed ABABAB and an HV therapy has not yet occurred, then AI model 410 (see FIG. 4) can provide a clinician with the predicted risk probability for that patient at 5 years. The clinician may inquire AI model 410, “If a certain type of device is programmed with BBBBAB and has had HV therapy, what is the risk at 9 years?” and receive an updated response, with a predicted risk probability calculated specifically for a patient with the risk estimated at 9 years.

According to certain examples, there are 64 programmable pathways and 64*2*10=1,280 scenarios for the example programmable pathways described above (e.g., ABABAB or BBBBAB), if risk is quantified on a yearly basis for 10 years.

A clinician may inquire, “If a device is programmed “AAAAAA” and when the first HV shock has an event, what's the chance of cumulative therapy failure by the end of the second shock, third shock, . . . and the sixth shock?” AI model 410 may responsively generate a response utilizing the trained Bayesian network structure to provide analysis specifying, for example: There are 64*(6+15+20+15+6)=64*62=3,968 scenarios for the programming pathway and occurrence described by the clinician inquiry.

A clinician may inquire, “What is the chance of all 6 shocks having events in an episode (i.e., 6 events in a roll in one episode), when the device is programmed ABABAB?” AI model 410 may responsively generate a response utilizing the trained Bayesian network structure to provide analysis specifying, for example: There are 64 scenarios matching the conditions described by the clinician inquiry.

The above scenarios may be analyzed by AI model 410 responsive to clinician inquiries for specific patients. Theoretically, there could be at least 64\hat{ }3*2*3*2*10=31,457,280 distinct scenarios simulated using the Bayesian network structure to quantify risk for different device/situations assuming there are 64 programmable pathways, 64 states of events at Rx1 to Rx6, 64 states of HV therapy success/failure at Rx1 to Rx6, HVT vs. non-HVT treatments, 3 different product types, 2 different leads, and a 10 year risk duration when risk is quantified at a yearly basis for each of the 10 years. Calculating risk for these 31 million+scenarios without use of the Bayesian network structure may take more than 310 million computation hours. The Bayesian network structure enables AI model 410 to quickly provide customized risk assessment for specified device inputs for a patient and/or health event observations associated with a patient to aid clinicians with health care related decision making for their patients.

According to certain examples, the Bayesian network structure may be trained pre-learned conditional probability tables, return projected efficacy, efficacy risk and all other conditional probabilities, joint probabilities and/or marginal probabilities, and updated using clinician inputs about particular patients based on different clinician needs to support healthcare related decision making for individual patients.

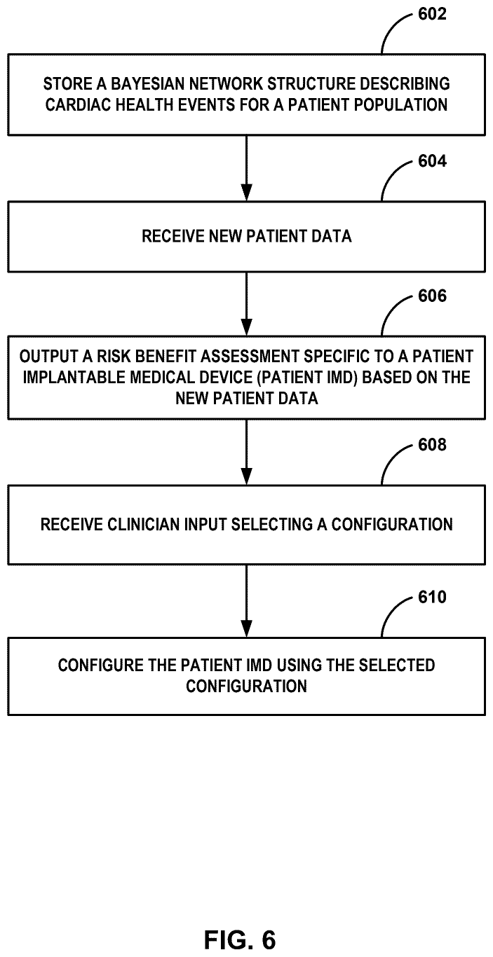

FIG. 6 is a flow diagram illustrating an example technique for evaluating patient risk associated with IMDs using Bayesian networks, in accordance with aspects of the disclosure. FIG. 6 is described with respect to processing system 100 and IMD 150 of FIG. 1, IMDs 250A-B of FIG. 2, processing system 300 of FIG. 3, external processing system 380 of FIG. 3, and the methods described in the context of FIGS. 4 and 5. However, the techniques of FIG. 6 may be performed by different components of processing system 100, external device 105, or by additional or alternative systems.

Memory 360 of processing system 300 may be configured to store a Bayesian network structure describing cardiac health events for a patient population (602). For instance, memory 360 of processing system 300 may be configured to store cardiac data associated with a patient population within a structure of Bayesian network 342, 396, wherein the cardiac data describes cardiac health events for the patient population.

According to such an example, processing circuitry 110 may be configured to receive new patient data 417 (604). For instance, processing circuitry may be configured to receive new patient data describing at least one of a new cardiac health event 369 determined by a patient implantable medical device (IMD) 150, a record of an arrhythmia treatment applied by the patient IMD 150, or a programming IMD configuration 106 for the patient IMD.

Continuing with such an example, processing circuitry 110 may be configured to output a risk benefit assessment specific 423A-C to a patient implantable medical device 450 based on the new patient data 417 (606). For instance, processing circuitry may be configured to output a risk-benefit assessment 423A-C specific to the patient IMD 450, wherein the risk-benefit assessment 423A-C indicates multiple recommended configurations 421A-C for the patient IMD 450 and a corresponding risk probability 422A-C for each of the multiple recommended configurations 421A-C based at least in part on application of the new patient data 417 to an Artificial Intelligence model (AI model) 410 trained using the Bayesian network structure 415 to describe cardiac health profiles for a patient population.

Processing circuitry may also be configured to receive clinician input selecting a configuration (608). For instance, subsequent to output of the risk-benefit assessment 423A-C specific to the patient IMD 450, processing circuitry may be configured to receive clinician input with specifying a selected configuration 445 from one of the multiple recommended configurations 421A-C for the patient IMD 450.

In some examples, processing circuitry 110 is configured to configure the patient IMD 450 using selected configuration 445. For instance, responsive to receipt of the clinician input specifying selected configuration 445 from the multiple recommended configurations 421A-C for the patient IMD 450, processing circuitry may be configured to configure delivery of a therapy via the patient IMD 450 using selected configuration 445 from the multiple recommended configurations 421A-C selected according to the clinician input.

Various aspects of the techniques may enable the following examples.
Example 1—A system comprising: a memory configured to store cardiac data associated with a patient population within a Bayesian network structure, wherein the cardiac data describes cardiac health events for the patient population; and processing circuitry in communication with the memory, wherein the processing circuitry is configured to: receive new patient data describing at least one of a new cardiac health event determined by a patient implantable medical device (IMD), a record of an arrhythmia treatment applied by the patient IMD, or a programming configuration for the patient IMD; output a risk-benefit assessment specific to the patient IMD, wherein the risk-benefit assessment indicates multiple configuration recommendations for the patient IMD and a corresponding risk probability for each of the multiple configuration recommendations based at least in part on application of the new patient data to an Artificial Intelligence model (AI model) trained using the Bayesian network structure to describe cardiac health profiles for a patient population; subsequent to output of the risk-benefit assessment specific to the patient IMD, receive clinician input with a selection of one of the multiple configuration recommendations for the patient IMD; and responsive to receipt of the clinician input with the selection of the one of the multiple configuration recommendations for the patient IMD, configure delivery of a therapy via the patient IMD using the one of the multiple configuration recommendations selected according to the clinician input.
Example 2—The system of example 1, wherein the processing circuitry is further configured to: recursively update the Bayesian network structure using the new patient data until the new patient data is represented within the Bayesian network structure.
Example 3—The system of example 2, wherein to recursively update the Bayesian network structure using the new patient data includes the processing circuitry further configured to: update one or more conditional probability tables of the Bayesian network structure using the new patient data; and output the risk-benefit assessment specific to the patient IMD using the one or more conditional probability tables updated using the new patient data.
Example 4—The system of any one of examples 1-3, wherein the Bayesian network structure describes the cardiac health profiles for the patient population based on existing clinical trial data and existing field data.
Example 5—The system of any one of examples 1-4, wherein the cardiac health profiles include one or more of: arrhythmia incidents within the patient population; arrhythmia treatment outcomes for the arrhythmia incidents within the patient population; IMD programming configurations for the patient population; or IMD product device types utilized by the patient population.
Example 6—The system of any one of examples 1-5, wherein the processing circuitry is further configured to: output the AI model, wherein the AI model is trained to generate patient-specific risk analysis for a patient based on the new patient data; and wherein the patient-specific risk analysis specifics at least one of: risk probability for the patient using the patient IMD when surgically implanted; risk probability for the patient using the patient IMD when configured with the one of the multiple configuration recommendations selected according to the clinician input; and risk probability for the patient using the patient IMD when configured using the programming configuration previously utilized for the patient IMD.
Example 7—The system of any one of examples 1-6, wherein the processing circuitry is further configured to: output the risk-benefit assessment specific to the patient IMD by applying methods including Maximum Likelihood Estimation (MLE) to the Bayesian network structure to identify the multiple configuration recommendations for the patient IMD and the corresponding risk probability for each of the multiple configuration recommendations probabilities based on frequencies observed in the new patient data within the Bayesian network structure.
Example 8—The system of any one of examples 1-7, wherein the patient IMD includes an implantable cardioverter defibrillator (ICD) type medical device.
Example 9—The system of example 8, wherein the programming configuration for the patient IMD includes ICD parameters including one or more of: ICD arrhythmias detection thresholds; ICD high-energy electrical shock sequencing; ICD high-energy electrical shock timing; ICD high-energy electrical shock vectors; ICD arrhythmia therapy repetition parameters; ICD arrhythmia therapy success thresholds; or ICD arrhythmia therapy failure thresholds.
Example 10—The system of example 8, wherein the processing circuitry is further configured to: train the AI model to generate as output, the risk-benefit assessment specific to the ICD type medical device, wherein to train the AI model includes the processing circuitry further configured to: obtain a priori conditional probability tables representing existing clinical trial data and the existing field; obtain training input parameters including observed health events within a subset of the patient population determined to have the ICD type medical device; train the AI model to integrate the new patient data into the a priori conditional probability tables; and update the AI model using the observed health events within the subset of the patient population determined to the ICD type medical device.
Example 11—The system of any one of examples 1-7, wherein the patient IMD includes an implantable cardiac resynchronization therapy defibrillator (CRT-D) type medical device.
Example 12—The system of example 11, wherein the programming configuration for the patient IMD includes CRT-D parameters including one or more of: CRT-D therapy vectors; CRT-D therapy timing intervals; CRT-D electrical stimulation vectors; CRT-D electrical stimulation magnitude; or CRT-D electrical stimulation delivery timing.
Example 13—The system of example 11, wherein to train the AI model to generate as output, the risk-benefit assessment specific to the CRT-D type medical device, includes the processing circuitry further configured to: obtain a priori conditional probability tables representing existing clinical trial data and the existing field; obtain training input parameters including observed health events within a subset of the patient population determined to have the CRT-D type medical device; train the AI model to integrate the new patient data into the a priori conditional probability tables; and update the AI model using the observed health events within the subset of the patient population determined to the CRT-D type medical device.
Example 14—The system of any one of examples 1-8 and 12, wherein to train the AI model to generate as output, the risk-benefit assessment specific to the patient IMD, includes the processing circuitry further configure to: update the AI model based on a high voltage therapy (HVT) treatment configuration previously programmed into the patient IMD; output the risk-benefit assessment specific to the patient IMD including outputting from the updated AI model, one or more of: projected efficacy and efficacy risk of the one of the multiple configuration recommendations selected according to the clinician input; a listing of conditional probabilities, joint probabilities, marginal probabilities, or some combination thereof utilized by the Bayesian network structure in determining the corresponding risk probability for the one of the multiple configuration recommendations selected according to the clinician input; a comparison of the corresponding risk probability for the one of the multiple configuration recommendations selected according to the clinician input with a subset of the patient population determined based on one or more overlapping health characteristics with a patient to receive the patient IMD determined based at least in part on the clinician input; and a risk over time projection for a configurable design-life of the patient IMD.
Example 15—The system of example 14, wherein the one or more overlapping health characteristics are selected from the group comprising: age; gender; medical diagnoses; risk factors; comorbidities; prior arrhythmia events; or HVT therapy prescriptions.
Example 16—The system of any one of examples 1-7, wherein each of the multiple configuration recommendations for the patient IMD specifies one or more general therapy programmable pathways for the patient IMD including any of: medical device product; biventricular pacing timing; pacing timing intervals; cardiac health event detection thresholds; or sensitivity thresholds for detecting patient physiological markers.
Example 17—The system of any one of examples 1-7, wherein each of the multiple configuration recommendations for the patient IMD specifies one or more Implantable Cardioverter Defibrillator-High Voltage (ICD-HV) programmable pathways for the patient IMD including any of: medical device product; quantity of electrical leads; type of the electrical leads; high-voltage therapy (HVT) treatment prescription; conditional HVT treatment prescription following one or more failed HVT treatment deliveries; or termination conditions for HVT treatment delivery.
Example 18—The system of any one of examples 1-7, wherein each of the multiple configuration recommendations for the patient IMD specifies one or more Cardiac Resynchronization Therapy (CRT) programmable pathways for the patient IMD including any of: medical device product; non-defibrillation electrical pulsing prescription; pulse generator type; electrical lead configuration; pulse timing; pulse intensity; pulse initiation thresholds; or pulse termination thresholds.
Example 19—A method comprising: receiving new patient data describing at least one of a new cardiac health event determined by a patient implantable medical device (IMD), a record of an arrhythmia treatment applied by the patient IMD, or a programming configuration for the patient IMD; outputting a risk-benefit assessment specific to the patient IMD identifying multiple configuration recommendations for the patient IMD and a corresponding risk probability for each of the multiple configuration recommendations based at least in part on application of the new patient data to an Artificial Intelligence model (AI model) trained using a Bayesian network structure to describe cardiac health profiles for a patient population; subsequent to outputting the risk-benefit assessment specific to the patient IMD, receiving clinician input selecting one of the multiple configuration recommendations for the patient IMD; and responsive to receiving the clinician input selecting the one of the multiple configuration recommendations for the patient IMD, configuring delivery of a therapy via the patient IMD using the one of the multiple configuration recommendations selected according to the clinician input.
Example 20—The method of example 1, further comprising: recursively updating the Bayesian network structure using the new patient data until the new patient data is represented within the Bayesian network structure.
Example 21—The system of example 20, wherein recursively updating the Bayesian network structure using the new patient data includes: updating one or more conditional probability tables of the Bayesian network structure using the new patient data; and outputting the risk-benefit assessment specific to the patient IMD using the one or more conditional probability tables updated using the new patient data.
Example 22—The method of any one of examples 19-21, wherein the Bayesian network structure describes the cardiac health profiles for the patient population based on existing clinical trial data and existing field data.
Example 23—The method of any one of examples 19-22, wherein the cardiac health profiles include one or more of: arrhythmia incidents within the patient population; arrhythmia treatment outcomes for the arrhythmia incidents within the patient population; IMD programming configurations for the patient population; or IMD product device types utilized by the patient population.
Example 24—The method of any one of examples 19-23, further comprising: outputting the AI model, wherein the AI model is trained to generate patient-specific risk analysis for a patient based on the new patient data; and wherein the patient-specific risk analysis specifies at least one of: risk probability for the patient using the patient IMD when surgically implanted; risk probability for the patient using the patient IMD when configured with the one of the multiple configuration recommendations selected according to the clinician input; and risk probability for the patient using the patient IMD when configured using the programming configuration previously utilized for the patient IMD.
Example 25—The method of any one of examples 19-24, further comprising: outputting the risk-benefit assessment specific to the patient IMD by applying methods including Maximum Likelihood Estimation (MLE) to the Bayesian network structure to identify the multiple configuration recommendations for the patient IMD and the corresponding risk probability for each of the multiple configuration recommendations probabilities based on frequencies observed in the new patient data within the Bayesian network structure.
Example 26—The method of any one of examples 19-25, wherein the patient IMD includes an implantable cardioverter defibrillator (ICD) type medical device.
Example 27—The method of example 26, wherein the programming configuration for the patient IMD includes ICD parameters including one or more of: ICD arrhythmias detection thresholds; ICD high-energy electrical shock sequencing; ICD high-energy electrical shock timing; ICD high-energy electrical shock vectors; ICD arrhythmia therapy repetition parameters; ICD arrhythmia therapy success thresholds; or ICD arrhythmia therapy failure thresholds.
Example 28—The method of example 26, wherein the AI model is trained to generate as output, the risk-benefit assessment specific to the ICD type medical device by the following operations: obtaining a priori conditional probability tables representing existing clinical trial data and the existing field; obtaining training input parameters including observed health events within a subset of the patient population determined to have the ICD type medical device; training the AI model to integrate the new patient data into the a priori conditional probability tables; and updating the AI model using the observed health events within the subset of the patient population determined to the ICD type medical device.
Example 29—The method of any one of examples 19-26, wherein the patient IMD includes an implantable cardiac resynchronization therapy defibrillator (CRT-D) type medical device.
Example 30—The method of example 29, wherein the programming configuration for the patient IMD includes CRT-D parameters including one or more of: CRT-D therapy vectors; CRT-D therapy timing intervals; CRT-D electrical stimulation vectors; CRT-D electrical stimulation magnitude; or CRT-D electrical stimulation delivery timing.
Example 31—The method of example 29, wherein the AI model is trained to generate as output, the risk-benefit assessment specific to the CRT-D type medical device, according to the following operations: obtaining a priori conditional probability tables representing existing clinical trial data and the existing field; obtaining training input parameters including observed health events within a subset of the patient population determined to have the CRT-D type medical device; training the AI model to integrate the new patient data into the a priori conditional probability tables; and updating the AI model using the observed health events within the subset of the patient population determined to the CRT-D type medical device.
Example 32—The method of any one of examples 19-26 and 29, wherein the AI model is trained to generate as output, the risk-benefit assessment specific to the patient IMD, according to the following operations: updating the AI model based on a high voltage therapy (HVT) treatment configuration previously programmed into the patient IMD; outputting the risk-benefit assessment specific to the patient IMD including outputting from the updated AI model, one or more of: projected efficacy and efficacy risk of the one of the multiple configuration recommendations selected according to the clinician input; a listing of conditional probabilities, joint probabilities, marginal probabilities, or some combination thereof utilized by the Bayesian network structure in determining the corresponding risk probability for the one of the multiple configuration recommendations selected according to the clinician input; a comparison of the corresponding risk probability for the one of the multiple configuration recommendations selected according to the clinician input with a subset of the patient population determined based on one or more overlapping health characteristics with a patient to receive the patient IMD determined based at least in part on the clinician input; and a risk over time projection for a configurable design-life of the patient IMD.
Example 33—The method of example 32, wherein the one or more overlapping health characteristics are selected from the group comprising: age; gender; medical diagnoses; risk factors; comorbidities; prior arrhythmia events; or HVT therapy prescriptions.
Example 34—The method of any one of examples 19-25, wherein each of the multiple configuration recommendations for the patient IMD specifies one or more general therapy programmable pathways for the patient IMD including any of: medical device product; biventricular pacing timing; pacing timing intervals; cardiac health event detection thresholds; or sensitivity thresholds for detecting patient physiological markers.
Example 35—The method of any one of examples 19-25, wherein each of the multiple configuration recommendations for the patient IMD specifies one or more Implantable Cardioverter Defibrillator-High Voltage (ICD-HV) programmable pathways for the patient IMD including any of: medical device product; quantity of electrical leads; type of the electrical leads; high-voltage therapy (HVT) treatment prescription; conditional HVT treatment prescription following one or more failed HVT treatment deliveries; or termination conditions for HVT treatment delivery.
Example 36—The method of any one of examples 19-25, wherein each of the multiple configuration recommendations for the patient IMD specifies one or more Cardiac Resynchronization Therapy (CRT) programmable pathways for the patient IMD including any of: medical device product; non-defibrillation electrical pulsing prescription; pulse generator type; electrical lead configuration; pulse timing; pulse intensity; pulse initiation thresholds; or pulse termination thresholds.
Example 37—A computer-readable medium comprising instructions to cause a processor to: store cardiac data associated with a patient population within a Bayesian network structure, wherein the cardiac data describes cardiac health events for the patient population; receive new patient data describing at least one of a new cardiac health event determined by a patient implantable medical device (IMD), a record of an arrhythmia treatment applied by the patient IMD, or a programming configuration for the patient IMD; output a risk-benefit assessment specific to the patient IMD, wherein the risk-benefit assessment indicates multiple configuration recommendations for the patient IMD and a corresponding risk probability for each of the multiple configuration recommendations based at least in part on application of the new patient data to an Artificial Intelligence model (AI model) trained using a Bayesian network structure to describe cardiac health profiles for a patient population; subsequent to output of the risk-benefit assessment specific to the patient IMD, receive clinician input with a selection of one of the multiple configuration recommendations for the patient IMD; and responsive to receipt of the clinician input with the selection of the one of the multiple configuration recommendations for the patient IMD, configure delivery of a therapy via the patient IMD using the one of the multiple configuration recommendations selected according to the clinician input.
Example 38—A computer program product having instructions to cause a processor to perform the method according to any one of examples 19-25.
Example 39—A system comprising means to perform the method according to any one of examples 19-25.

Various examples have been described. These and other examples are within the scope of the following claims.

What is claimed is:

1. A system comprising:
a memory configured to store cardiac data associated with a patient population within a Bayesian network structure, wherein the cardiac data describes cardiac health events for the patient population; and
processing circuitry in communication with the memory, wherein the processing circuitry is configured to:
receive new patient data describing at least one of a new cardiac health event determined by a patient implantable medical device (IMD), a record of an arrhythmia treatment applied by the patient IMD, or a programming configuration for the patient IMD;
output a risk-benefit assessment specific to the patient IMD, wherein the risk-benefit assessment indicates multiple configuration recommendations for the patient IMD and a corresponding risk probability for each of the multiple configuration recommendations based at least in part on application of the new patient data to an Artificial Intelligence model (AI model) trained using the Bayesian network structure to describe cardiac health profiles for a patient population;
subsequent to output of the risk-benefit assessment specific to the patient IMD, receive clinician input with a selection of one of the multiple configuration recommendations for the patient IMD; and
responsive to receipt of the clinician input with the selection of the one of the multiple configuration recommendations for the patient IMD, configure delivery of a therapy via the patient IMD using the one of the multiple configuration recommendations selected according to the clinician input.

2. The system of claim 1, wherein the processing circuitry is further configured to:
recursively update the Bayesian network structure using the new patient data until the new patient data is represented within the Bayesian network structure.

3. The system of claim 2, wherein to recursively update the Bayesian network structure using the new patient data includes the processing circuitry further configured to:
update one or more conditional probability tables of the Bayesian network structure using the new patient data; and
output the risk-benefit assessment specific to the patient IMD using the one or more conditional probability tables updated using the new patient data.

4. The system of claim 1, wherein the Bayesian network structure describes the cardiac health profiles for the patient population based on existing clinical trial data and existing field data.

5. The system of claim 1, wherein the cardiac health profiles include one or more of:
arrhythmia incidents within the patient population;
arrhythmia treatment outcomes for the arrhythmia incidents within the patient population;
IMD programming configurations for the patient population; or
IMD product device types utilized by the patient population and device data.

6. The system of claim 1, wherein the processing circuitry is further configured to:
output the AI model, wherein the AI model is trained to generate patient-specific risk analysis for a patient based on the new patient data; and
wherein the patient-specific risk analysis specifies at least one of:
risk probability for the patient using the patient IMD when surgically implanted;
risk probability for the patient using the patient IMD when configured with the one of the multiple configuration recommendations selected according to the clinician input; and
risk probability for the patient using the patient IMD when configured using the programming configuration previously utilized for the patient IMD.

7. The system of claim 1, wherein the processing circuitry is further configured to:
output the risk-benefit assessment specific to the patient IMD by applying methods including Maximum Likelihood Estimation (MLE) to the Bayesian network structure to identify the multiple configuration recommendations for the patient IMD and the corresponding risk probability for each of the multiple configuration recommendations probabilities based on frequencies observed in the new patient data within the Bayesian network structure.

8. The system of claim 1, wherein the patient IMD includes an implantable cardioverter defibrillator (ICD) type medical device.

9. The system of claim 8, wherein the programming configuration for the patient IMD includes ICD parameters including one or more of:
ICD arrhythmias detection thresholds;
ICD high-energy electrical shock sequencing;
ICD high-energy electrical shock timing;
ICD high-energy electrical shock vectors;
ICD arrhythmia therapy repetition parameters;
ICD arrhythmia therapy success thresholds; or
ICD arrhythmia therapy failure thresholds.

10. The system of claim 8, wherein the processing circuitry is further configured to:
train the AI model to generate as output, the risk-benefit assessment specific to the ICD type medical device, wherein to train the AI model includes the processing circuitry further configured to:
obtain a priori conditional probability tables representing existing clinical trial data and the existing field;
obtain training input parameters including observed health events within a subset of the patient population determined to have the ICD type medical device;
train the AI model to integrate the new patient data into the a priori conditional probability tables; and
update the AI model using the observed health events within the subset of the patient population determined to the ICD type medical device.

11. The system of claim 1, wherein the patient IMD includes an implantable cardiac resynchronization therapy defibrillator (CRT-D) type medical device.

12. The system of claim 11, wherein the programming configuration for the patient IMD includes CRT-D parameters including one or more of:
CRT-D therapy vectors;
CRT-D therapy timing intervals;
CRT-D electrical stimulation vectors;
CRT-D electrical stimulation magnitude; or
CRT-D electrical stimulation delivery timing.

13. The system of claim 11, wherein to train the AI model to generate as output, the risk-benefit assessment specific to the CRT-D type medical device, includes the processing circuitry further configured to:
obtain a priori conditional probability tables representing existing clinical trial data and the existing field;
obtain training input parameters including observed health events within a subset of the patient population determined to have the CRT-D type medical device;
train the AI model to integrate the new patient data into the a priori conditional probability tables; and
update the AI model using the observed health events within the subset of the patient population determined to the CRT-D type medical device.

14. The system of claim 1, wherein to train the AI model to generate as output, the risk-benefit assessment specific to the patient IMD, includes the processing circuitry further configure to:
update the AI model based on a high voltage therapy (HVT) treatment configuration previously programmed into the patient IMD;
output the risk-benefit assessment specific to the patient IMD including outputting from the updated AI model, one or more of:
projected efficacy and efficacy risk of the one of the multiple configuration recommendations selected according to the clinician input;
a listing of conditional probabilities, joint probabilities, marginal probabilities, or some combination thereof utilized by the Bayesian network structure in determining the corresponding risk probability for the one of the multiple configuration recommendations selected according to the clinician input;
a comparison of the corresponding risk probability for the one of the multiple configuration recommendations selected according to the clinician input with a subset of the patient population determined based on one or more overlapping health characteristics with a patient to receive the patient IMD determined based at least in part on the clinician input; and
a risk over time projection for a configurable design-life of the patient IMD.

15. A method comprising:
receiving new patient data describing at least one of a new cardiac health event determined by a patient implantable medical device (IMD), a record of an arrhythmia treatment applied by the patient IMD, or a programming configuration for the patient IMD;
outputting a risk-benefit assessment specific to the patient IMD identifying multiple configuration recommendations for the patient IMD and a corresponding risk probability for each of the multiple configuration recommendations based at least in part on application of the new patient data to an Artificial Intelligence model (AI model) trained using a Bayesian network structure to describe cardiac health profiles for a patient population;
subsequent to outputting the risk-benefit assessment specific to the patient IMD, receiving clinician input selecting one of the multiple configuration recommendations for the patient IMD; and
responsive to receiving the clinician input selecting the one of the multiple configuration recommendations for the patient IMD, configuring delivery of a therapy via the patient IMD using the one of the multiple configuration recommendations selected according to the clinician input.

16. The method of claim 15, further comprising:
recursively updating the Bayesian network structure using the new patient data until the new patient data is represented within the Bayesian network structure; and
wherein recursively updating the Bayesian network structure using the new patient data includes:
updating one or more conditional probability tables of the Bayesian network structure using the new patient data; and
outputting the risk-benefit assessment specific to the patient IMD using the one or more conditional probability tables updated using the new patient data.

17. The method of claim 15, further comprising:
outputting the AI model, wherein the AI model is trained to generate patient-specific risk analysis for a patient based on the new patient data; and
wherein the patient-specific risk analysis specifies at least one of:
risk probability for the patient using the patient IMD when surgically implanted;
risk probability for the patient using the patient IMD when configured with the one of the multiple configuration recommendations selected according to the clinician input; and
risk probability for the patient using the patient IMD when configured using the programming configuration previously utilized for the patient IMD.

18. The method of claim 15:
wherein the patient IMD includes an implantable cardiac resynchronization therapy defibrillator (CRT-D) type medical device; and
wherein the AI model is trained to generate as output, the risk-benefit assessment specific to the CRT-D type medical device, according to the following operations:
obtaining a priori conditional probability tables representing existing clinical trial data and the existing field;
obtaining training input parameters including observed health events within a subset of the patient population determined to have the CRT-D type medical device;
training the AI model to integrate the new patient data into the a priori conditional probability tables; and
updating the AI model using the observed health events within the subset of the patient population determined to the CRT-D type medical device.

19. The method of claim 15, wherein the AI model is trained to generate as output, the risk-benefit assessment specific to the patient IMD, according to the following operations:
updating the AI model based on a high voltage therapy (HVT) treatment configuration previously programmed into the patient IMD;
outputting the risk-benefit assessment specific to the patient IMD including outputting from the updated AI model, one or more of:
projected efficacy and efficacy risk of the one of the multiple configuration recommendations selected according to the clinician input;
a listing of conditional probabilities, joint probabilities, marginal probabilities, or some combination thereof utilized by the Bayesian network structure in determining the corresponding risk probability for the one of the multiple configuration recommendations selected according to the clinician input;
a comparison of the corresponding risk probability for the one of the multiple configuration recommendations selected according to the clinician input with a subset of the patient population determined based on one or more overlapping health characteristics with a patient to receive the patient IMD determined based at least in part on the clinician input; and
a risk over time projection for a configurable design-life of the patient IMD.

20. A computer-readable medium comprising instructions to cause a processor to:
store cardiac data associated with a patient population within a Bayesian network structure, wherein the cardiac data describes cardiac health events for the patient population;
receive new device and patient data describing at least one of a new cardiac health event determined by a patient implantable medical device (IMD), a record of an arrhythmia treatment applied by the patient IMD, or a programming configuration for the patient IMD;
output a risk-benefit assessment specific to the patient IMD, wherein the risk-benefit assessment indicates multiple configuration recommendations for the patient IMD and a corresponding risk probability for each of the multiple configuration recommendations based at least in part on application of the new patient data to an Artificial Intelligence model (AI model) trained using a Bayesian network structure to describe cardiac health profiles for a patient population;
subsequent to output of the risk-benefit assessment specific to the patient IMD, receive clinician input with a selection of one of the multiple configuration recommendations for the patient IMD; and
responsive to receipt of the clinician input with the selection of the one of the multiple configuration recommendations for the patient IMD, configure delivery of a therapy via the patient IMD using the one of the multiple configuration recommendations selected according to the clinician input.
